(19) United States
(12) Patent Application Publication
(10) Pub. No.: US 2023/0320674 A1
(43) Pub. Date: Oct. 12, 2023

(54) MONITORING ASSISTANCE SYSTEM AND MONITORING ASSISTANCE METHOD

(71) Applicants: YUTAKA ELECTRONICS CO., LTD., Hashima-shi, Gifu (JP); GREED LTD., Gifu-shi, Gifu (JP)

(72) Inventors: Yutaka AONO, Hashima-shi (JP); Futoshi IMAMICHI, Gifu-shi (JP); Chong Kun YOO, Bucheon-shi (KR)

(73) Assignees: YUTAKA ELECTRONICS CO., LTD., Hashima-shi, Gifu (JP); GREED LTD., Gifu-shi, Gifu (JP)

(21) Appl. No.: 18/022,165

(22) PCT Filed: Aug. 27, 2020
(86) PCT No.: PCT/JP2020/032359

§ 371 Date: Feb. 19, 2023

(51) Int. Cl. A61B 5/00; H04N 23/66; A61B 5/024

(52) U.S. Cl. CPC A61B 5/746; A61B 5/681; A61B 2562/08; A61B 5/02438; H04N 23/66

(57) ABSTRACT

Vital data including data regarding a pulse is continuously obtained from a wearable terminal worn by a person to be monitored and stored in a database as target quantitative information associated with disease information and physical condition information regarding the person; an analysis with time-series patterns based on the vital data as an explanatory variable and the disease information and/or the physical condition information at and after times when the time-series patterns were obtained as a response variable is performed using the target quantitative information accumulated in the database; a time-series pattern based on new vital data is compared with the time-series pattern found in the analysis to be correlated with the disease information and/or the physical condition information; and a physical event including disease onset and a physical condition change of a person who has shown the new vital data is estimated or predicted.

### BACKGROUND OF THE INVENTION

### Field of the invention

The present invention relates to a monitoring assistance system and a monitoring assistance method.

### Description of the Related Art

Even if there are signs before onset of disease or a sudden change in a medical condition, a person is often unaware of these signs. This is especially true for the elderly, as their physical senses are dull. Even if a person feels that he/she is not in good physical condition, it is often difficult for the person to properly communicate to a medical care worker what is wrong with him/her. Especially in the case of a patient with dementia, even a medical interview is difficult where the patient is asked about his/her physical condition and symptoms. In addition, although it is recommended that blood pressure and body temperature be taken at regular times each day to monitor one's own physical condition and maintain health, many people are unable to continue to do so. A person's inability to “notice”, “communicate”, or “continue” might thus delay detection of physical abnormalities and make diseases more serious.

Wearable terminals that can be worn on a wrist like wristwatches to measure a pulse rate, body temperature, and the like non-invasively without attaching electrodes to a human body have been proposed (e.g., PTL 1). Such wearable terminals are utilized, for example, by health-conscious people for their own health management. During sports, such people adjust a load of exercise on the basis of changes in a pulse rate or grasp the amount of exercise on the basis of the number of steps taken.

The present inventors thought that such wearable terminals could be used to detect physical abnormalities at early stages by having elderly or sick people wear the wearable terminals. Conventional wristwatch-type wearable terminals are designed for temporary use, such as during sports, and elaborate designs thereof regarding monitor indications result in large power consumption. Because a battery needs to be frequently charged, such wearable terminals cannot be continuously used. The present inventors, on the other hand, have suppressed power consumption by devising a data measurement method and an output format, thereby making it possible to use such wearable terminals continuously for a long period of at least one week without charging a battery. This makes it possible to continuously obtain data (vital data) regarding a body of a person who wears the wearable terminal. With a conventional wearable terminal, however, a pulse rate, body temperature, and the like are only displayed on a monitor of the wearable terminal. It is therefore necessary for a person to consciously read values and monitor changes in the values, and it is difficult for elderly or sick people to perform such tasks.

### CITATION LIST

### Patent Document

Patent Document 1: Japanese Patent Application Laid-open No. 2017-18236

### SUMMARY OF THE INVENTION

In view of the above circumstances, the present invention aims to provide a monitoring assistance system and a monitoring assistance method capable of detecting physical abnormalities at early stages by obtaining vital data regarding a person who wears a wearable terminal without requiring the person to perform any tasks.

In order to solve the above problem, the monitoring assistance system (hereinafter also simply referred to as a “system”) in the present invention includes:

“a wearable terminal that is worn by a person to be monitored and that continuously obtains vital data including data regarding a pulse;

a watcher terminal associated with the wearable terminal; and

a management server communicably connected to at least one of the watcher terminal or the wearable terminal,

in which at least one of the watcher terminal or the management server includes first vital data processing means for receiving the vital data to which an identification code of the wearable terminal is attached and performing data processing on the vital data,

in which the first vital data processing means includes

vital change detection means for detecting, on a basis of a change in the vital data, a physical event including disease onset and a physical condition change, and

alert means for generating an alert signal on a basis of the detection performed by the vital change detection means and notifying the watcher terminal of occurrence of an abnormality, and

in which the management server includes a database storing target information, in which the person to be monitored is associated with the wearable terminal, and target quantitative information, in which the vital data obtained by the wearable terminal is associated with the target information”.

In the monitoring assistance system, a wearable terminal worn by a person to be monitored continuously obtains vital data including data regarding a pulse. As described in detail later, physical events including disease onset and physical condition changes, such onset of atrial fibrillation, awakening during sleep, fever, a sign of a pulse stop, and a pulse stop, manifest as changes in the vital data. In the monitoring assistance system, first vital data processing means, which is a functional component of the system, detects physical events by detecting changes in the vital data obtained continuously and notifies a watcher terminal of occurrence of abnormalities on the basis of alert signals. Here, the watcher terminal is a terminal used by a watcher, who monitors a person to be monitored, and examples of the watcher include medical care workers (hereinafter referred to as “medical workers”), visiting medical care workers such as visiting nurses and visiting care workers (hereinafter referred to as “visiting medical workers”), and family members of the person.

The system can therefore obtain vital data regarding a person who wears a wearable terminal, detect a physical abnormality at an early stage through data processing therein, and notify a watcher of the abnormality without requiring the person to perform any tasks. As a result, physical abnormalities that in the past would have not been detected promptly due to a person's inability to notice, communicate, or continue can be identified at early stages, and appropriate measures can be promptly taken. Diseases, therefore, can be kept from becoming serious.

In addition, the vital data is stored in a database of a management server while being associated with target information regarding the person to be monitored. The vital data, which is difficult to store in the wearable terminal or the watcher terminal that is expected to be carried around by the watcher, because the vital data is continuously obtained and the amount thereof becomes huge as time passes, therefore, can be stored for a long time without being deleted after single use. The “continuous” obtaining of the vital data refers to accumulation of daily obtaining of data at regular time intervals or at certain times (regular times).

In addition to the above configuration, the monitoring assistance system in the present invention may further include:

“a camera apparatus that is communicably connected to the watcher terminal and that is provided in a living space of the person to be monitored,

in which the camera apparatus may enter a monitoring state on a basis of the transmission of the alert signal by the alert means and signal transmission from the watcher terminal”.

With this configuration, image-based communication can be performed between a camera apparatus provided in the vicinity of the person to be monitored and the watcher terminal. When the camera apparatus enters a monitoring state on the basis of transmission of an alert signal by alert means, therefore, the watcher can immediately visually check a state of the person after being notified of occurrence of an abnormality. When the camera apparatus enters the monitoring state on the basis of signal transmission from the watcher terminal, on the other hand, the watcher can check the state of the person via the camera apparatus at any time when he/she desires to know how the person is doing. It is of course possible for the watcher, upon being notified of occurrence of an abnormality, to immediately transmit a signal for placing the camera apparatus in the monitoring state. In addition, when the watcher is a family member of the person, for example, the watcher can have a sense of security since he/she can see the person at any time.

In the above configuration of the monitoring assistance system in the present invention, “disease information and physical condition information may be included as the target information for each person to be monitored,

the disease information and the physical condition information may be updated on a basis of an input from the watcher terminal,

the management server may include second vital data processing means for performing data processing using the target quantitative information accumulated in the database, and

the second vital data processing means may include

time-series pattern analysis means for conducting an analysis with time-series patterns based on the vital data in past as an explanatory variable and at least one of the disease information or the physical condition information at and after times when the time-series patterns were obtained as a response variable and finding a correlation, and

event estimation and prediction means for comparing a time-series pattern based on new vital data with the time-series pattern found by the time-series pattern analysis means to be correlated with at least one of the disease information or the physical condition information and estimating or predicting a physical event including disease onset and a physical condition change of a person to be monitored who has shown the new vital data”.

With this configuration, data processing is performed using target quantitative information accumulated in the database. That is, the data processing is performed using a huge amount of vital data obtained over a long period of time for a large number of persons to be monitored. The target quantitative information is associated with the target information including disease information and physical condition information. Any abnormality that occurs in a body is considered to be correlated with some variable in the disease information or the physical condition information. The second vital data processing means in the system, therefore, conducts an analysis with time-series patterns, which are graphical patterns of temporal changes in the vital data, as an explanatory variable, and at least one of the disease information or the physical condition information at and after the time-series patterns were obtained as a response variable, and finds a correlation.

If a past time-series pattern is found, as a result of this process, to be correlated with some variable in the disease information or the physical condition information, the past time-series pattern can be used to estimate or predict an abnormality in a body of a person who has shown new vital data. That is, if a time-series pattern based on the new vital data is similar to the past time-series pattern, it can be estimated or predicted that a physical event corresponding to at least one of the disease information or the physical condition information found to be correlated with the past time-series pattern has occurred or will occur in the body of the person who has shown the new vital data. If the disease information and/or the physical condition information found to be correlated with the past time-series pattern is from the same point in time as when the past time-series pattern was obtained, “estimation” of a current abnormality is performed, and if the disease information and/or the physical condition information found to be correlated with the past time-series pattern is from a point in time after the past time-series pattern was obtained, “prediction” of an abnormality that will occur in the future is performed. In the analysis conducted by the time-series pattern analysis means, the disease information prior to (before) the time when the past time-series pattern was obtained can be added as an explanatory variable.

Next, a monitoring assistance method in the present invention includes:

“continuously obtaining vital data including data regarding a pulse from a wearable terminal worn by a person to be monitored;

storing the obtained vital data in a database as target quantitative information associated with disease information and physical condition information regarding the person to be monitored;

performing, using the target quantitative information accumulated in the database, an analysis process for conducting, through machine learning, an analysis with time-series patterns based on the vital data as an explanatory variable and at least one of the disease information or the physical condition information at and after times when the time-series patterns were obtained as a response variable; and

comparing a time-series pattern based on new vital data with the time-series pattern found in the analysis process to be correlated with at least one of the disease information or the physical condition information and estimating or predicting a physical event including disease onset and a physical condition change of a person to be monitored who has shown the new vital data”.

This configuration is a monitoring assistance method used by the monitoring assistance system, which performs data processing using the target quantitative information accumulated in the database. The processing performed by the second vital data processing means is processing performed through machine learning.

In the above configuration of the monitoring assistance method in the present invention,

“the time-series pattern based on the new vital data may be a temporal fluctuation pattern of a pulse rate, and

a time of a pulse stop of the person to be monitored who has not been given life-support measures may be predicted as the physical event”.

As described in detail later, with this method, a time of a pulse stop can be predicted for a person to be monitored who has not been given life-support measures, and the person's close relatives can prepare themselves to care for the person in his/her final days. In addition, even when the person lives on his/her own, the watcher, if he/she is a visiting medical worker or a close relative, is notified of a sign of a pulse stop and the pulse stop, and a situation where the person dies unnoticed can be avoided. Here, the “life-support measures” can be exemplified by administration of drugs for maintaining the vital data within a normal range, such as antihypertensive and hypertensive drugs, oxygen inhalation, and use of a heart-lung machine.

As described above, according to the present invention, a monitoring assistance system and a monitoring assistance method capable of detecting physical abnormalities at early stages by obtaining vital data regarding a person who wears a wearable terminal without requiring the person to perform any tasks can be provided.

### BRIEF DESCRIPTION OF THE DRAWINGS

FIG. 1 is a diagram illustrating the configuration of a monitoring assistance system according to an embodiment of the present invention.

FIG. 2 is a graph showing temporal changes in a pulse rate and skin temperature at a time of awakening at night.

FIG. 3A is a graph showing temporal changes in a pulse rate and skin temperature of a healthy person at rest, and FIG. 3B is a graph showing temporal changes in a pulse rate and skin temperature of a patient with atrial fibrillation at rest.

FIG. 4A is a graph showing temporal changes in a pulse rate and skin temperature of a bedridden person who is not given life-support measures one to two weeks before a pulse stops, and FIG. 4B is a graph showing temporal changes in the pulse rate and the skin temperature of the same person on a day when death was declared.

### DESCRIPTION OF THE EMBODIMENTS

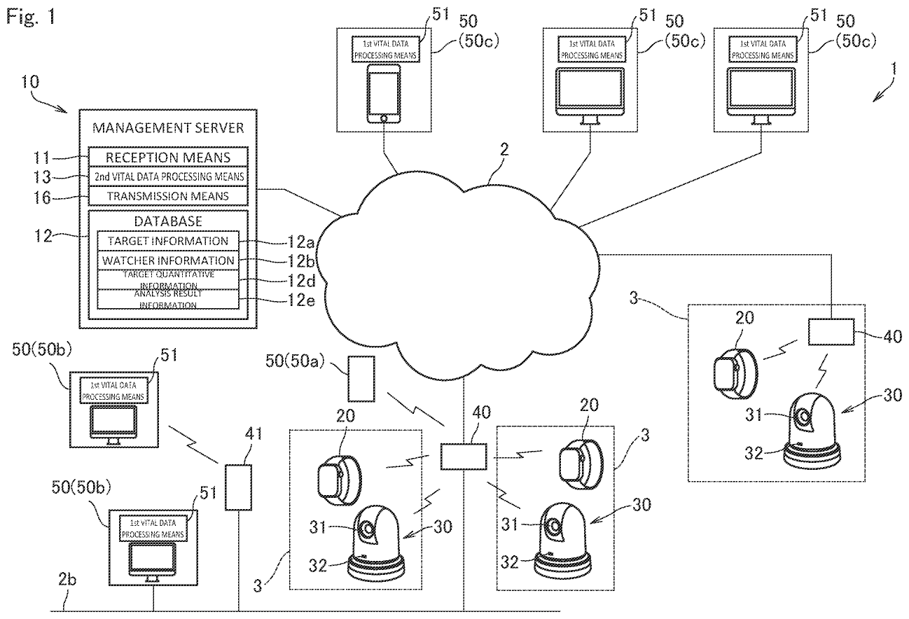

A monitoring assistance system 1 according to a specific embodiment of the present invention will be described hereinafter with reference to the drawings. Persons to be monitored by the monitoring assistance system 1 according to the present embodiment include residents of nursing homes, patients in hospitals, and persons who live at home but need medical care or nursing care. The monitoring assistance system 1 according to the present embodiment includes, as FIG. 1 illustrates a schematic configuration thereof, a management server 10, wearable terminals 20, camera apparatuses 30, dedicated transceivers 40, and watcher terminals 50.

The wearable terminal 20 is a terminal worn on a wrist of a person to be monitored like a wristwatch and includes a CPU, a storage device, and a monitor. The wearable terminal 20 obtains data regarding a pulse, skin temperature, the number of steps taken (activity level), blood pressure, and SpO₂ (oxygen saturation level) as vital data regarding the persons. Among these measurement items, the data regarding a pulse, the skin temperature, and the number of steps taken are constantly obtained. “Constantly” here refers to continuous obtaining of data at short intervals of one to two minutes.

In the vital data, the blood pressure and SpO₂ may also be constantly obtained, or may be obtained at certain time intervals or at certain times. Here, the “certain time intervals” may be set as, for example, four hours or six hours. The “certain times” may be set as regular times of every day such as 7:00 (upon getting out of bed), 12:00 (before lunch), and 20:00 (before going to bed). The measurement items to be obtained at the certain time intervals or at the certain times may be obtained at any times on the basis of signals transmitted from the watcher terminal 50, instead.

The data regarding a pulse is a pulse rate and PPIs and measured by photoelectric pulse wave measuring method. Photoelectric pulse wave measuring method is roughly divided into transmission pulse wave measurement, in which the amount of change in light transmitted through a body is measured, and reflection pulse wave measurement, in which the amount of change in light reflected within a body is measured. In the present embodiment, reflection pulse wave measurement using a reflection pulse wave sensor is employed. More specifically, when volume of blood vessels changes due to pulsation, the amount of hemoglobin in blood also changes. Since hemoglobin absorbs green light, pulsation, and thus a pulse wave, can be detected by utilizing a fact that reflected light radiated by green LEDs onto the blood vessels varies depending on the amount of hemoglobin. The PPIs are peak intervals (in seconds) of a pulse waveform, and the pulse rate is an average PPI per minute (60 divided by an average PPI). In the present embodiment, HRV (heart rate variability) is also measured as data regarding a pulse in addition to the pulse rate and the PPIs.

The skin temperature is measured by detecting, using a thermistor, changes in electrical resistance due to temperature changes. The number of steps taken (activity level) is measured by measuring, using a three-axis acceleration sensor, the number of times that three-axis acceleration has acted. With respect to the blood pressure, because volume of blood vessels increases as the blood pressure increases, and accordingly the amount of hemoglobin increases, the pulse wave is detected on the basis of reflected light of light emitted from the green LEDs as in the reflection pulse wave measurement, and a blood pressure value is estimated by measuring pulse propagation from a shape of the pulse wave. SpO₂ is calculated on the basis of changes in intensity of the reflected light of the light emitted from the LEDs.

The wearable terminal 20 uses a battery as a power supply, but by devising a method for measuring vital data and changing information to be displayed on a monitor, the wearable terminal 20 consumes significantly less power than a conventional wristwatch-type wearable terminal, and can be used continuously for a long period of one to two weeks. Even if an elderly person who lives alone visited by a visiting medical worker only once or twice a week is the person to be monitored, therefore, it is sufficient for the visiting medical worker to replace the battery during the visit. Vital data can be continuously obtained without requiring the person himself/herself to replace the battery. A battery level may be transmitted to a watcher terminal 50 associated with the wearable terminal 20.

The camera apparatus 30 is provided in a living space 3 of the person to be monitored who wears the wearable terminal 20. The camera apparatus 30 includes a camera 31, a microphone 32, and a speaker (not illustrated). The camera apparatus 30 outputs voices transmitted from the watcher terminal 50 from the speaker, collects sounds therearound, and transmits the sounds to the watcher terminal 50. That is, the camera apparatus 30 is a remote camera through which conversation can take place. The camera apparatus 30 can turn on and off the camera 31 and the microphone 32 on the basis of signals transmitted from the watcher terminal 50 and change a shooting direction and shooting magnification of the camera 31.

The dedicated transceiver 40 is provided in the living space 3 of the person to be monitored who wears the wearable terminal 20 or a building including the living space 3. The dedicated transceiver 40 is a computer including a modem with a router function in addition to a CPU and a storage device and connected to the wearable terminal 20 and the camera apparatus 30 through wireless communication such as Wi-Fi or Bluetooth (registered trademark) and to a communication network 2 such as the Internet by wire. When persons to be monitored live in a facility such as a nursing home or a hospital, for example, the dedicated transceiver 40 may be provided in a living space 3 of each of the persons, each of floors of the facility, or for a certain number of living spaces 3.

The watcher terminal 50 is a terminal used by a watcher, who monitors the person to be monitored. The watcher may be, for example, a medical worker, a visiting medical worker, a family member of the person. When the person to be monitored is a resident of a nursing home, for example, the watcher terminal 50 may be, for example, a terminal used by a medical worker who looks after the resident or a terminal provided for an office of the nursing home. When the person is a patient in a hospital, the watcher terminal 50 may be, for example, a terminal used by a medical worker such as a doctor or a therapist, such as a terminal used by a nurse who looks after the person or a terminal provided in a nurse station. When the person to be monitored is a person who lives at home but needs medical care or nursing care, the watcher terminal 50 may be, for example, a terminal used by a visiting medical worker who looks after the person or a terminal at an office to which the visiting medical worker belongs.

The watcher terminal 50 is a computer including a CPU, a storage device, input devices such as a keyboard and a mouse, and output devices such as a monitor and a printer. The watcher terminal 50 according to the present embodiment also includes a microphone and a speaker. The watcher's voice can be collected by the microphone and transmitted to the camera apparatus 30, and sounds transmitted from the camera apparatus 30 can be output from the speaker. The watcher terminal 50 may be a desktop personal computer, a laptop personal computer, a tablet computer, or a smartphone.

Dedicated software for using the monitoring assistance system 1 is installed on the watcher terminal 50. As a result, the watcher terminal 50 includes reception means, transmission means (both are not illustrated), and first vital data processing means 51 as functional components. The watcher terminal 50 communicates data and signals with the dedicated transceiver 40 and the management server 10 through the reception means and the transmission means.

In the present embodiment, the watcher terminals 50 include watcher terminals 50a that wirelessly communicate with the dedicated transceivers 40 and that are connected to the communication network 2 through the dedicated transceivers 40, watcher terminals 50b that communicate with the dedicated transceivers 40 by wire over a facility communication network 2b and that are connected to the communication network 2 through the dedicated transceivers 40, and watcher terminals 50c connected to the communication network 2 without using the dedicated transceivers 40. The watcher terminals 50a and 50b, which are provided in the same facilities as the wearable terminals 20, can communicate data and signals with the wearable terminals 20 through the dedicated transceivers 40 without using the communication network 2. The watcher terminals 50a, 50b, and 50c are referred to as “watcher terminals 50” herein when the watcher terminals 50a, 50b, and 50c need not be particularly distinguished from one another.

The first vital data processing means 51 is means for immediately performing data processing on vital data obtained by the wearable terminal 20. The first vital data processing means 51 includes vital change detection means and alert means. The vital change detection means is means for detecting, on the basis of changes in the vital data, physical events including disease onset and physical condition changes of the person to be monitored. The physical events including disease onset and physical condition changes will be described in detail later.

The alert means is means for causing the watcher terminal 50 to notify of occurrence of abnormalities on the basis of detection by the vital change detection means. The notification by the watcher terminal 50 may be lighting up or blinking of a warning light, display of a warning on a monitor screen, outputting of a warning sound from the speaker, or a combination of some or all of these.

The management server 10 is a server managed by a manager of the monitoring assistance system 1 and connected to the communication network 2. The management server 10 is a computer including a CPU, a storage device, input devices such as a keyboard and a mouse, and output devices such as a monitor and a printer. The management server 10 includes reception means 11, a database 12, second vital data processing means 13, and transmission means 16 as functional components.

The management server 10 communicates, using the reception means 11 and the transmission means 16, data and signals with the watcher terminals 50a and 50b through the dedicated transceivers 40. The management server 10 also communicates, using the reception means 11 and the transmission means 16, with the watcher terminals 50c without using the dedicated transceivers 40.

The second vital data processing means 13 is means for performing data processing using target quantitative information 12d that has been accumulated for a certain period of time, the target quantitative information 12d being vital data obtained by the wearable terminals 20, associated with target information 12a, and stored in the database 12. The second vital data processing means 13 includes status information generation means, time-series pattern analysis means, and event estimation and prediction means.

The database 12 stores the target information 12a, watcher information 12b, the target quantitative information 12d, and analysis result information 12e. The target information 12a is information in which information such as gender and age (date of birth) of the persons to be monitored is associated with identification codes of the wearable terminals 20 worn by the persons. The target information 12a according to the present embodiment also includes information in which an identification code of a dedicated transceiver 40 to which each of the wearable terminals 20 transmits vital data and an identification code of a camera apparatus 30 provided in a living space 3 in which the wearable terminal 20 is provided are associated with the identification code of the wearable terminal 20. The target information 12a according to the present embodiment also includes disease information and physical condition information regarding the persons to be monitored. The disease information and the physical condition information will be described later.

The watcher information 12b is information in which the watcher terminals 50 and the wearable terminals 20 are associated with each other using identification codes thereof. The watcher information 12b also includes authentication information for the watcher terminals 50 to access the management server 10, such as IDs and passwords.

The watcher terminals 50 and the wearable terminals 20 associated with each other need not necessarily be in one-to-one correspondence. When a plurality of medical workers and visiting medical workers look after one person to be monitored, for example, a plurality of watcher terminals 50 and one wearable terminal 20 are associated with each other. When one medical worker or visiting medical worker looks after a plurality of persons to be monitored, one watcher terminal 50 and a plurality of wearable terminals 20 are associated with each other.

The target quantitative information 12d is information in which vital data obtained by the wearable terminals 20 is associated with the target information 12a. The target quantitative information 12d may also include results of data processing performed on vital data using the first vital data processing means 51. The analysis result information 12e is information in which, if a correlation is found between a time-series pattern and at least one of disease information or physical condition information as a result of an analysis conducted by the time-series pattern analysis means in the data processing performed by the second vital data processing means 13, the disease information and/or the physical condition information is associated with the time-series pattern and that is stored in the database 12.

Next, a monitoring assistance method employing the monitoring assistance system 1 according to the present embodiment will be described. First, a person to be monitored wears the wearable terminal 20. The camera apparatus 30 is installed in a living space 3 of the person, and the dedicated transceiver 40 is installed in the living space 3 or a building including the living space 3.

In the watcher terminal 50, dedicated software for using the monitoring assistance system 1 is installed and activated. The watcher terminal 50 logs in to the management server 10 by inputting authentication information over the communication network 2. As a result, the watcher terminal 50 can read the watcher information 12b, and find the wearable terminal 20 associated therewith. In addition, when the person to be monitored changes from day to day, for example, the watcher information 12b can be updated by associating, on the basis of an input from the watcher terminal 50, the watcher terminal 50 with a wearable terminal 20 worn by a new person to be monitored. When the watcher information 12b is updated, the management server 10 registers a watcher terminal 50 newly associated with a dedicated transceiver 40 associated with the new wearable terminal 20. That is, the dedicated transceiver 40 that receives vital data from the wearable terminal 20 obtains information indicating a destination of the vital data (transmission to a watcher terminal 50a, transmission to a watcher terminal 50b, or transmission to a watcher terminal 50c through the management server 10). Alternatively, the dedicated transceiver 40 may obtain, on the basis of an input from a watcher terminal 50a or 50b provided in the same facility as the wearable terminal 20 without using the management server 10, information regarding the watcher terminal 50a or 50b to which the dedicated transceiver 40 is to transmit the vital data.

When use of the monitoring assistance system 1 starts in this state, the wearable terminal 20 transmits the vital data obtained thereby with an identification code thereof attached to the vital data. When the wearable terminal 20 is associated with the watcher terminal 50a or 50b used by the watcher in the same facility such as a nursing home or a hospital, the dedicated transceiver 40 that has received the vital data from the wearable terminal 20 transmits the vital data to the watcher terminal 50a or 50b without using the communication network 2. At this time, another transceiver 41 may mediate the data transmission when the dedicated transceiver 40 that has received the vital data from the wearable terminal 20 transmits the vital data to the watcher terminal 50a or 50b.

Upon receiving the vital data, the watcher terminal 50a or 50b performs data processing using the first vital data processing means 51. In the data processing, first, the vital change detection means detects changes in the vital data and, if it is determined that the changes indicate an abnormality, the alert means generates an alert signal and transmits the alert signal to notification devices (the speaker, the warning light, and the monitor) of the watcher terminal 50a or 50b. As a result, the watcher terminal 50a or 50b notifies of occurrence of the abnormality.

The alert signal is also transmitted to the dedicated transceiver 40 that has mediated the transmission of the vital data and the camera apparatus 30 associated with the wearable terminal 20 worn by the person to be monitored in whom the abnormality has occurred. The camera apparatus 30 switches the camera 31 to a monitoring state and the microphone 32 to a sound collection state on the basis of the reception of the alert signal. As a result, the watcher who is using the watcher terminal 50a or 50b notified of the occurrence of the abnormality can immediately visually recognize, through the camera 31, a state of the person to be monitored and have a conversation by talking to the person through the speaker and listening to the person through the microphone 32. The watcher can then rush to the person in accordance with the recognized state.

When the person to be monitored and the watcher are in the same facility as described above, the vital data transmitted from the wearable terminal 20 is transmitted to the watcher terminal 50a or 50b without using the communication network 2 and subjected to the data processing. The data processing, therefore, can be performed in almost real-time and, if an abnormality occurs in the person, the watcher can respond extremely quickly.

The vital data received by the watcher terminal 50a or 50b is transmitted to the management server 10 over the communication network 2 at a certain timing along with a result of data processing performed by the first vital data processing means 51 and stored in the database 12 as target quantitative information 12d. A large amount of vital data, therefore, need not be stored in a storage device of the watcher terminal 50a or 50b.

When the person to be monitored and the watcher are geographically distant from each other, that is, when the person to be monitored is at home and the watcher is a visiting medical worker, on the other hand, the vital data transmitted from the wearable terminal 20 is transmitted by the dedicated transceiver 40 to the management server 10 over the communication network 2. Upon receiving the vital data, the management server 10 transmits the vital data to a watcher terminal 50c associated with the wearable terminal 20. Data processing in the watcher terminal 50c that has received the vital data is the same as that described above for the watcher terminal 50a or 50b.

Now, the data processing performed by the vital change detection means included in the first vital data processing means 51 will be described while taking examples. The vital change detection means detects physical events including disease onset and physical condition changes through a process in which changes in vital data are detected and compared with a predetermined threshold or a process in which changes in vital data in a plurality of measurement items are combined together. Here, the “physical events including disease onset and physical condition changes” to be detected refer to occurrence of abnormalities in a body (transition to states different from before) exemplified by awakening during sleep (mid-sleep awakening), onset of atrial fibrillation, anemia or heat stroke and associated syncope, fever, a sign of a pulse stop, and a pulse stop.

### Awakening during Sleep (Mid-Sleep Awakening) and Getting out of Bed

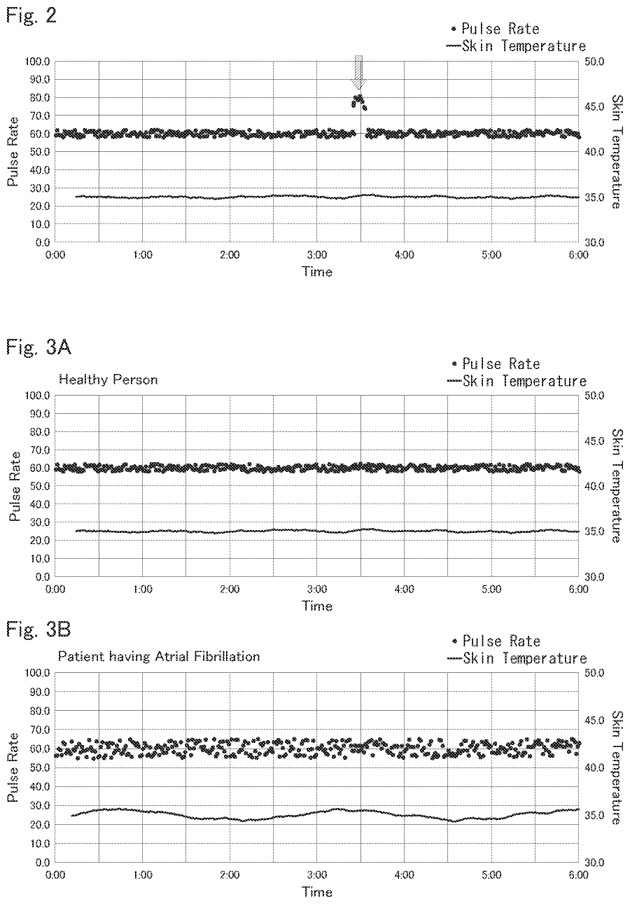

FIG. 2 is a graph where a pulse rate and skin temperature during sleep are plotted against time. The pulse rate during sleep is stable at low values, but temporarily increases upon a mid-sleep awakening (indicated by an arrow in the figure). By comparing the pulse rate in the vital data continuously obtained by the wearable terminal 20 with a pulse rate a certain period of time ago (e.g., three to five minutes ago), therefore, it can be determined that there has been a mid-sleep awakening (detection of a physical event) if a difference (the amount of change) remains larger than or equal to a certain threshold (e.g., 15 to 20) for a certain period of time (e.g., two to three minutes) or longer. If a further increase in the pulse rate is detected and the number of steps taken is measured thereafter, it can be detected, on the basis of these, that the person to be monitored has gotten out of bed and begun to move.

When a person to be monitored, such as an elderly person, awakes at night and wanders or goes to a bathroom alone, the person might fall and break a bone or injure himself/herself. If the person breaks a bone, for example, a level of care required increases, and burdens on both the person and a medical worker increase. In order to avoid such an event, medical workers make frequent nighttime rounds at nursing homes and hospitals, which places a heavy burden on the medical workers. There is also a system that monitors movement of a person to be monitored by installing a camera that captures an image of scene around a bed of the person and processing the captured image. In this case, however, the system detects the movement only after the person awakes and gets out of bed. By a time a medical worker is notified on the basis of the detection, therefore, the person might not be there, or has fallen down at a place where he/she has gone. In order to avoid such problems, some facilities administer sleeping pills to keep persons to be monitored from waking up at night, but there is concern that this might increase a physical burden on the persons.

In view of these problems, the watcher terminal 50 is notified of occurrence of an abnormality as soon as a person to be monitored wakes up, that is, before the person gets out of bed, in the monitoring assistance system 1 according to the present embodiment. This makes it possible for a watcher to respond quickly by, for example, calling out, through the camera apparatus 30, to the person who has woken up before he/she starts to move, thereby preventing the person from wandering alone.

### Onset of Atrial Fibrillation

FIGS. 3A and 3B are graphs where resting pulse rates and skin temperature of a healthy person and a patient with atrial fibrillation, respectively, plotted against time. As can be seen from FIG. 3A, the pulse rate of the healthy person at rest is stable at low values, whereas, as illustrated in FIG. 3B, the pulse rate of the patient with atrial fibrillation does not stabilize even at rest and greatly varies in some periods of time. By detecting a difference between a maximum value and a minimum value of a pulse rate in vital data at rest, such as during sleep, in a certain period of time (e.g., 3 to 5 minutes), therefore, it can be determined that there has been onset of atrial fibrillation (detection of a physical event) if the difference remains larger than or equal to a certain threshold (e.g., 3 to 7) for a certain period of time (e.g., 10 to 30 minutes) or longer. In addition, when an atrial fibrillation occurs, a cycle of a pulse wave is more irregular than usual. Onset of atrial fibrillation, therefore, can be detected by comparing a standard deviation of PPIs with a certain threshold or by comparing HRV with a certain threshold.

Atrial fibrillation is an arrhythmia caused by abnormal electrical excitation in atria. Because the atria contract irregularly as if in spasm, a pulse rate irregularly varies. Blood is not pumped normally from within the atria, and blood clots tend to be formed. If an atrial fibrillation occurs frequently, cerebral infarction and dementia can be caused. Atrial fibrillation ca thus be a cause of serious diseases, but because it is painless and imperceptible, there is concern that detection thereof might be delayed. In addition, atrial fibrillation often remains undetected even by electrocardiograms occasionally taken at hospitals, because it is not known when it will manifest. It is said that continuous week-long electrocardiography is necessary to detect onset of atrial fibrillation with electrocardiograms, but such a large-scale examination is difficult to perform.

In view of these conventional problems, vital data constantly measured by the wearable terminal 20 in the monitoring assistance system 1 according to the present embodiment includes a pulse rate and PPIs (or HRV). Even onset of atrial fibrillation, which can occur at any time, therefore, can be detected with a high probability.

### Anemia or Heat Stroke and Associated Syncope

Blood pressure and a pulse rate rise and fall in tandem with each other when normal. When physical exercise is started, for example, blood pressure rises and a pulse rate also rises, and when physical exercise is stopped, blood pressure decreases and returns to a normal value, and the pulse rate also decreases and returns to a normal value. In contrast, when anemia occurs or syncope occurs as a result of anemia, blood pressure suddenly decreases while a pulse rate increases. A difference between values of blood pressure and a pulse rate in vital data at the same point in time, therefore, may be detected, and if the difference is larger than a certain threshold, that is, if the blood pressure and the pulse rate do not change in tandem with each other, it can be determined that a person is prone to anemia or associated syncope (detection of a physical event). If the pulse rate increases in discrepancy from the blood pressure and skin temperature increases above a certain threshold, it can be determined that a person is prone to heat stroke and associated syncope (detection of a physical event).

### Fever

When the number of steps taken (activity level) in vital data is close to zero (within a range of 0 to “0+ a certain threshold”), a person is considered to be at rest. If, in this state, it is detected, as a result of detection of changes in the vital data, that both a pulse rate and skin temperature have increased above their respective certain thresholds, it can be determined that the increase in the pulse rate and body temperature is not a healthy one due to physical exercise but a fever caused by a disease (detection of a physical event).

### Sign of Pulse Stop and Pulse Stop

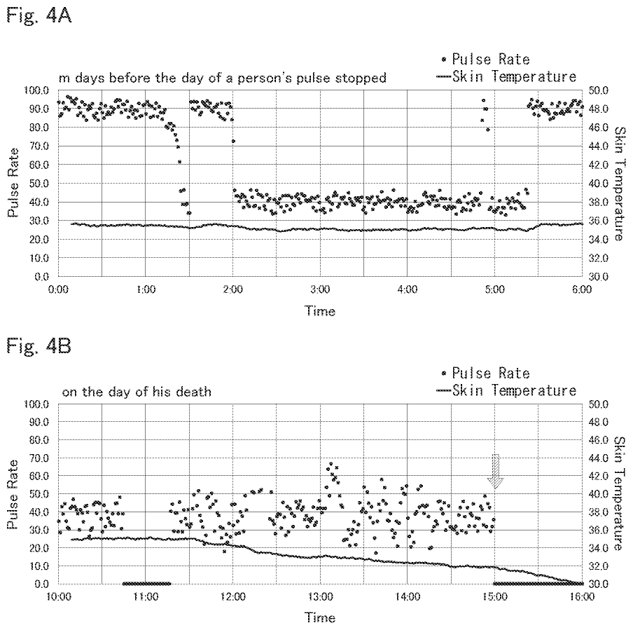

It has been conventionally thought that when a person dies quietly in a bedridden state without taking life-support measures, a pulse rate is substantially constant as during sleep and gradually decreases toward an end of life. Contrary to this conventional common wisdom of those skilled in the art, the present inventors have found, as illustrated in FIG. 4A, that, even in a bedridden state, a pulse rate greatly varies in a block-like manner one to two weeks before a pulse stops (a period in which the pulse rate continues to be high and a period in which the pulse rate continues to be low repeat irregularly). On a day when death is declared, the pulse rate, which has become low, repeats irregular fluctuation at short intervals and the skin temperature decreases until the pulse stops as illustrated in FIG. 4B (indicated by an arrow in the figure).

With the monitoring assistance system 1 according to the present embodiment, therefore, if it is detected that a pulse rate has remained lower than or equal to a certain threshold (e.g., 39) for a certain period of time (e.g., 10 to 15 minutes) or longer, it is determined that a pulse will stop before long (a sign of a pulse stop) and an alert signal is generated. The watcher terminal 50 then notifies of occurrence of an abnormality. As a result, a medical worker and family members contacted by the medical worker can, without delay, care for an end of life of a person to be monitored. In addition, if it is detected in the monitoring assistance system 1 that a pulse rate has remained zero for a certain period of time (e.g., 30 to 45 minutes) or longer, it is determined that a pulse has stopped, and an alert signal is generated. The watcher terminal 50 then notifies of occurrence of an abnormality. As a result, a situation where a person to be monitored dies unnoticed can be avoided even when the person is geographically distant from a visiting medical worker or a family member who is a watcher and does not see the watcher every day, such as when the person lives on his/her own. A pulse stop is one of conditions under which a doctor declares death. A doctor declares death when heartbeat stops and pupils become dilated in addition to a pulse stop.

A case where physical events are detected in almost real-time through the immediate data processing performed by the first vital data processing means 51 has been described above. Next, the processing performed by the second vital data processing means 13, that is, the data processing performed using the target quantitative information 12d accumulated in the database 12 for a certain period of time, will be described.

For this processing, target information 12a for each person to be monitored includes disease information and physical condition information. The disease information is information regarding a name of a disease that the person is suffering from, such as dementia, diabetes, or angina pectoris, and may be composed of a name of a disease in one of major categories such as a brain disease, a cardiac disease, and a respiratory disease and a name of a disease in one of more specific subcategories included in the major category. The physical condition information is information in which events that have occurred in relation to a physical condition of the person are associated with times at which the events have occurred and frequencies. The “events that have occurred in relation to a physical condition” include events that can be recognized by a third party, such as coughing, dizziness, vomiting, falling, and coma and events complained by the person, such as a chest pain and a headache. As described above, the physical condition information also includes mid-sleep awakening, anemia or heat stroke and associated syncope, fever, a sign of a pulse stop, and a pulse stop detected by the first vital data processing means as described above, and the disease information also includes a detected disease such as atrial fibrillation.

The disease information and the physical condition information may be registered to the database 12 on the basis of inputs from the watcher terminal 50 and updated on a daily basis. The software that is dedicated for the monitoring assistance system 1 and that is installed on the watcher terminal 50 displays candidates for the disease information and the physical condition information and times and frequencies to be included in the physical condition information on the monitor as a pull-down menu. Necessary information, therefore, can be easily input just by selecting an item on the pull-down menu, without inputting information as text. The disease information and the physical condition information stored in the database 12 can be output as necessary using a watcher terminal 50 connected to the management server 10 and used as records that replace care and nursing records.

Status information generation means included in the second vital data processing means 13 generates status information in a short period of time in the past for each person to be monitored. For example, vital data in 24 hours of a previous day is subjected to data processing in units of measurement items, and averages, maximum values, minimum values, and the like of the measurement items are calculated as status information. If there has been a notification about occurrence of an abnormality based on detection of a physical event on the day, a type of physical event is identified (screening) on the basis of conditions of data processing in which the detection has been performed and included in the status information. At the same time, the identified physical event is added to the disease information or the physical condition information included in the target quantitative information 12d. Death is also added to the physical condition information as a physical event along with a time of the death.

The management server 10 transmits the generated status information to a watcher terminal 50 associated with the person to be monitored, and a monitor of the watcher terminal 50 displays the status information. For example, status information from a previous day may be transmitted every day at a fixed time in the morning. If an abnormality has not been detected on the previous day, information indicating the absence of an abnormality may be included in the status information.

The time-series pattern analysis means included in the second vital data processing means 13 is means for performing data processing with a large amount of target quantitative information 12d regarding a large number of persons to be monitored stored in the database 12. The huge amount of target quantitative information 12d accumulated (big data) includes a graph indicating temporal changes in vital data of each of the measurement items. Patterns of changes in these graphs are “time-series patterns”. The time-series patterns are associated with the disease information and the physical condition information, but it is unclear which variables, among a large number of variables included in the disease information and the physical condition information, the time-series patterns affect. Since the time-series patterns are data patterns based on vital data regarding humans, however, it is considered that the time-series patterns correlate with the disease information and the physical condition information regarding physical events of the humans.

A process for conducting an analysis with time-series patterns as an explanatory variable and at least one of disease information or physical condition information after times when the corresponding time-series patterns were obtained as a response variable and finding a correlation, therefore, is performed. The process is performed through machine learning. If a correlation between a time-series pattern and a variable in the disease information or the physical condition information is found as a result of the analysis, the time-series pattern and the variable are stored in the database 12 while being associated with each other as analysis result information 12e. Disease information before (preceding) times when the corresponding time-series patterns were obtained may also be added as an explanatory variable. In this case, the time-series patterns are analyzed while taking into consideration diseases that persons had already known to suffered from.

The event estimation and prediction means included in the second vital data processing means 13 performs data processing on new vital data using analysis result information 12e created on the basis of past vital data and estimates or predicts a physical event for a person to be monitored who has shown the new vital data. That is, a time-series pattern based on the new vital data is compared with a past time-series pattern included in the analysis result information 12e, and if it is determined that the two patterns are similar to each other, it can be estimated or predicted that a physical event found to be correlated with the past time-series pattern (onset of a disease indicated by correlated disease information or a physical condition change indicated by correlated physical condition information) will occur to the person who has shown the new vital data.

Here, disease information or physical condition information found to be correlated with a past time-series pattern with which the disease information or the physical condition information has been compared is information “after” the time-series pattern was obtained. If the disease information or the physical condition information is “concurrent” with the time-series pattern, however, it is estimated that a corresponding physical event has already occurred to a person who has shown new vital data. Even if the person who has shown the new vital data has no subjective symptoms or a medical worker is unaware of the physical event, a detailed examination can be performed with reference to this estimation, and early treatment can be provided.

If disease information or physical condition information found to be correlated with a past time-series pattern with which the disease information or the physical condition has been compared is information “after a time” when the time-series pattern was obtained, it is predicted that a corresponding physical event will occur in future to a person who has shown new vital data. If a correlation has been found between a past time-series pattern indicating that an atrial fibrillation had occurred at a certain frequency during a certain period of time and disease information based on a physical event of “onset of stroke in n days”, and if it is determined that the past time-series pattern and a time-series pattern based on new vital data are similar to each other, for example, it is predicted that a person who has shown the new vital data will have a stroke in the near future of about n days. As a result, appropriate medical measures can be taken before the onset of the stroke, thereby preventing the onset of the stroke or preventing symptoms from becoming severe. Medical and social security costs, therefore, can be significantly reduced.

In addition, as illustrated in FIG. 4A, a graph showing temporal changes in a pulse rate of a person whose “pulse stopped after m days” has been obtained. If time-series patterns similar to the graph are obtained for a certain percentage (X %) or more of those declared dead within a certain number of days (n days), a correlation between the time-series patterns and physical condition information based on a physical event of “a pulse stop within n days” is recognized and included in the analysis result information 12e. If it is determined that such past time-series patterns and a time-series pattern based on new vital data are similar to each other, it is predicted that a pulse of a person who has shown the new vital data will stop in the near future of about n days with a probability of X %. As a result, the person's close relatives can prepare themselves to care for the person in his/her final days.

If some physical event is estimated or predicted as a result of the data processing performed by the event estimation and prediction means, the information may be included in the above-described status information.

Although the present invention has been described with reference to a preferred embodiment, the present invention is not limited to the above embodiment and may be improved or modified in various ways without departing from the aspect of the present invention.

Although the wearable terminal 20 is worn on a wrist like a wristwatch in the above embodiment, for example, the wearable terminal 20 is not limited to this. The wearable terminal 20 may be worn on another part of a human body, such as an ankle or an upper arm, instead.

In addition, although the first vital data processing means 51 is a functional component of the watcher terminal 50 in the above embodiment, the first vital data processing means 51 may be a functional component of the management server 10, instead. In this case, vital data transmitted from the wearable terminal 20 is transmitted to the management server 10 through the dedicated transceiver 40, and the management server 10 detects a physical event on the basis of changes in the vital data. If the management server 10 detects a physical event, the management server 10 transmits an alert signal to the watcher terminal 50, and the watcher terminals 50 notifies of occurrence of an abnormality.

Furthermore, in the above embodiment, a case has been described where the analysis result information 12e created by analyzing the huge amount of target quantitative information 12d accumulated in the database is used to estimate or predict a physical event on the basis of new vital data regarding a person to be monitored, who is a user of the monitoring assistance system 1. Usage of the analysis result information 12e in the database 12 is not limited to this, and the analysis result information 12e may be provided for a medical worker such as a doctor as a disease case library, instead. Since the analysis result information 12e is a database in which variables in the disease information and the physical condition information and the time-series patterns in the vital data found to be correlated with the variables are associated with each other, a current medical condition of a patient assigned to the medical worker such as a doctor can be estimated or future changes in the medical condition can be predicted by searching the database for and extracting a time-series pattern similar to that of the patient.

In addition, although a case where the vital data of each of the measurement items is subjected to data processing in order to detect, estimate, and predict physical events has been described in the above embodiment, skin temperature may be used to determine whether to use vital data of the other measurement items for data processing. That is, data obtained by the wearable terminal 20 while a person to be monitored is not wearing the wearable terminal 20, such as during bathing, does not reflect physical conditions. If a measured skin temperature is far from human skin temperature, therefore, other pieces of vital data measured at the time may be ignored. This can prevent false alert signals from being transmitted on the basis of data that does not reflect physical conditions.

What is claimed is:

1. A monitoring assistance system comprising:
a wearable terminal that is worn by a person to be monitored and that continuously obtains vital data including data regarding a pulse;
a watcher terminal associated with the wearable terminal; and
a management server communicably connected to at least one of the watcher terminal or the wearable terminal,
wherein at least one of the watcher terminal or the management server includes first vital data processing means for receiving the vital data to which an identification code of the wearable terminal is attached and performing data processing on the vital data,
wherein the first vital data processing means includes
vital change detection means for detecting, on a basis of a change in the vital data, a physical event including disease onset and a physical condition change, and
alert means for generating an alert signal on a basis of the detection performed by the vital change detection means and notifying the watcher terminal of occurrence of an abnormality, and
wherein the management server includes a database storing target information, in which the person to be monitored is associated with the wearable terminal, and target quantitative information, in which the vital data obtained by the wearable terminal is associated with the target information.

2. The monitoring assistance system according to claim 1, further comprising:
a camera apparatus that is communicably connected to the watcher terminal and that is provided in a living space of the person to be monitored,
wherein the camera apparatus enters a monitoring state on a basis of the transmission of the alert signal by the alert means and signal transmission from the watcher terminal.

3. The monitoring assistance system according to claim 1,
wherein disease information and physical condition information are included as the target information for each person to be monitored,
wherein the disease information and the physical condition information are updated on a basis of an input from the watcher terminal,
wherein the management server includes second vital data processing means for performing data processing using the target quantitative information accumulated in the database, and
wherein the second vital data processing means includes
time-series pattern analysis means for conducting an analysis with time-series patterns based on the vital data in past as an explanatory variable and at least one of the disease information or the physical condition information at and after times when the time-series patterns were obtained as a response variable and finding a correlation, and
event estimation and prediction means for comparing a time-series pattern based on new vital data with the time-series pattern found by the time-series pattern analysis means to be correlated with at least one of the disease information or the physical condition information and estimating or predicting a physical event including disease onset and a physical condition change of a person to be monitored who has shown the new vital data.

4. A monitoring assistance method comprising:
continuously obtaining vital data including data regarding a pulse from a wearable terminal worn by a person to be monitored;
storing the obtained vital data in a database as target quantitative information associated with disease information and physical condition information regarding the person to be monitored;
performing, using the target quantitative information accumulated in the database, an analysis process for conducting, through machine learning, an analysis with time-series patterns based on the vital data as an explanatory variable and at least one of the disease information or the physical condition information at and after times when the time-series patterns were obtained as a response variable; and
comparing a time-series pattern based on new vital data with the time-series pattern found in the analysis process to be correlated with at least one of the disease information or the physical condition information and estimating or predicting a physical event including disease onset and a physical condition change of a person to be monitored who has shown the new vital data.

5. The monitoring assistance method according to claim 4,
wherein the time-series pattern based on the new vital data is a temporal fluctuation pattern of a pulse rate, and
wherein a time of a pulse stop of the person to be monitored who has not been given life-support measures is predicted as the physical event.
